(19) United States
(12) Patent Application Publication
(10) Pub. No.: US 2015/0160231 A1
(43) Pub. Date: Jun. 11, 2015

(54) IDENTIFICATION OF METABOLITES FROM TANDEM MASS SPECTROMETRY DATA USING DATABASES OF PRECURSOR AND PRODUCT ION DATA

(71) Applicant: Premier Biosoft, Palo Alto, CA (US)

(72) Inventors: Ningombam Sanjib Meitei, Indore (m.p.) (IN); Arun Apte, San Francisco, CA (US)

(73) Assignee: Premier Biosoft, Palo Alto, CA (US)

(21) Appl. No.: 14/099,585

(22) Filed: Dec. 6, 2013

(51) Int. Cl. G01N 33/68

(52) U.S. Cl. CPC G01N 33/6848

(57) ABSTRACT

Techniques for identifying metabolites in a sample may utilize observed precursor and product ion data obtained by subjecting the sample to tandem mass spectrometry (MS/MS). The techniques may include first accessing a database of precursor ion data to identify one or more matching candidate metabolites in the database that match the observed precursor ion data. Each of the matching candidate metabolites may then be further validated by matching product ion data associated with the matching candidate metabolite and stored in a database of computationally generated product ion fragments with the observed product ion data comprising product ion fragments. A structure of a metabolite in the sample may be identified by selecting a candidate metabolite based on scores computed for the candidate metabolites based on the matching. Large volumes of metabolite precursor and product ion data may be analyzed in this way with improved speed and accuracy.

### BACKGROUND

1. Field of Invention

This application relates generally to identifying metabolites in a sample using precursor and product ion data obtained by subjecting the sample to mass spectrometry and more specifically to identifying metabolites by comparing experimentally obtained precursor and product ion data with computationally generated data.

2. Related Art

Mass spectrometry encompasses a broad range of techniques for identifying and characterizing compounds in mixtures. Different types of mass spectrometry-based approaches may be used to analyze a sample to determine its composition. For example, mass spectrometry in combination with a separation technique, such as liquid chromatography, is one of widely used mass spectrometric approaches.

Mass spectrometry analysis involves converting a sample being analyzed into multiple ions by an ionization process. Each of the resulting ions, when placed in a force field, moves in the field along a trajectory such that its acceleration is inversely proportional to its mass-to-charge ratio. A mass spectrum of a molecule is thus produced that displays a plot of relative abundances of precursor ions versus their mass-to-charge ratios. When a subsequent stage of mass spectrometry, such as tandem mass spectrometry, is used to further analyze the sample by subjecting precursor ions to higher energy, each precursor ion may undergo disassociation into fragments referred to as product ions. Resulting fragments can be used to provide information concerning the nature and the structure of their precursor molecule.

### BRIEF SUMMARY

Some embodiments provide techniques for structural identification of metabolites in a sample from data acquired by tandem mass spectrometry analysis of the sample. The data may comprise precursor and product data and that may be acquired experimentally, using any suitable mass spectrometry technique or a combination of mass spectrometry techniques. The experimentally acquired data may be compared to precursor and product ion data that may be generated computationally. A metabolite may then be identified in the sample based on the comparison.

Some embodiments relate to a computer-implemented method of identifying a metabolite in a sample. The method comprises operating at least one processor to: receive product ion data comprising observed product ion fragments generated by fragmenting a precursor ion having a first precursor m/z value using tandem mass spectrometry of the sample, each product ion fragment from the observed product ion fragments having a first product m/z value from a plurality of first product m/z values and an intensity value corresponding to the first product m/z value; compare the plurality of first product m/z values to at least one set of second product m/z values of second product ion fragments of a molecule associated with a second precursor m/z value that matches the first precursor m/z value to identify at least one matching set of second product ion fragments; and identify the metabolite based on the at least one matching set.

Some embodiments relate to at least one non-transitory computer-readable storage medium storing computer-executable instructions that, when executed by at least one processor, perform a method of identifying a metabolite in a sample. The method comprises receiving product ion data comprising observed product ion fragments generated by fragmenting a precursor ion having a first precursor m/z value using tandem mass spectrometry of the sample, each product ion fragment from the observed product ion fragments having a first product m/z value from a plurality of first product m/z values and an intensity value corresponding to the first product m/z value; comparing the plurality of first product m/z values to at least one set of second product m/z values of second product ion fragments of a molecule associated with a second precursor m/z value that matches the first precursor m/z value to identify at least one matching set of second product ion fragments; and identifying the metabolite based on the at least one matching set.

Some embodiments relate to a system comprising at least one processor and at least one storage medium having encoded thereon computer-executable instructions that, when executed by the at least one processor, cause the at least one processor to perform a method of identifying a metabolite in a sample. The method comprises receiving mass spectrum data obtained by analyzing the sample using a tandem mass spectrometer, the mass spectrum data comprising observed product ion fragments generated by fragmenting a precursor ion having a first precursor m/z value, each product ion fragment having a first product m/z value from a plurality of first product m/z values and an intensity value corresponding to the first product m/z value; comparing the plurality of first product m/z values to at least one set of second product m/z values of second product ion fragments of a molecule associated with a second precursor m/z value that matches the first precursor m/z value to identify at least one matching set of second product ion fragments; and identifying the metabolite based on the at least one matching set.

Some embodiments relate to a computing device comprising at least one processor, and memory communicatively coupled to the at least one processor, the memory configured to store a data structure comprising a plurality of entries, wherein: each entry from the plurality of entries stores first information on at least one product ion fragment of a metabolite that would result from fragmenting the metabolite using a tandem mass spectrometer; and each entry from the plurality of entries stores second information on the following: a value corresponding to an identifier of each product ion fragment from the at least one product ion fragment, a representation of a molecular structure of the product ion fragment, a representation of a molecular formula of the product ion fragment, and a mass of the product ion fragment.

Some embodiments relate to a computer-implemented method of identifying a metabolite in a sample. The method comprises operating at least one processor to: receive precursor ion data on an observed precursor ion and product ion data on observed product ion fragments obtained by subjecting a sample to tandem mass spectrometry, the product ion fragments generated by fragmenting the precursor ion by subjecting the sample to the tandem mass spectrometry; access a first database storing precursor ion data to identify at least one matching candidate metabolite that matches the observed precursor ion data; for each candidate metabolite of the at least one matching candidate metabolite, access a second database storing computationally generated product ion data to retrieve product ion fragments in the second database that are computationally generated for the candidate metabolite; compare the retrieved computationally generated product ion fragments to the observed product ion fragments; based on the comparison, identify at least one metabolite of the at least one matching candidate metabolite that is associated with computationally generated product ion fragments matching the observed product ion fragments; and identify the metabolite in the sample based on the at least one identified metabolite.

The foregoing summary is provided by way of illustration and not limitation.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a sketch of a computing system in which some embodiments of the invention may be implemented;

FIG. 2A is a schematic diagram illustrating information stored in a precursor ion data database, in accordance with some embodiments;

FIG. 2B is a schematic diagram illustrating information stored in a product ion data database, in accordance with some embodiments;

FIG. 3 is a flowchart illustrating a process of identifying a metabolite in a sample, in accordance with some embodiments;

FIG. 4 is another flowchart illustrating a process of identifying a metabolite in a sample, in accordance with some embodiments;

FIG. 5A is an illustration of examples of product ion spectra that may be analyzed using the techniques in accordance with some embodiments;

FIG. 5B is an illustration of an example of representing a product ion spectrum as a list of m/z and intensity values, in accordance with some embodiments;

FIGS. 6A and 6B are sketches illustrating a process of identifying a metabolite in a sample by accessing information in a product ion data database, in accordance with some embodiments;

FIG. 7 is a sketch of an MS/MS spectrum annotated based on results of identification of the metabolite using the process illustrated in FIGS. 6A and 6B, in accordance with some embodiments;

FIG. 8 is a flowchart illustrating a process of computing a score for a candidate metabolite, in accordance with some embodiments;

FIG. 9 depicts schematic diagrams illustrating a process of computing a score for a candidate metabolite, in accordance with some embodiments;

FIG. 10 is a sketch of an MS/MS spectrum obtained by subjecting a sample comprising an iodotyrosine compound to tandem mass spectrometry; and

FIG. 11 is a block diagram illustrating an exemplary computing device in which some embodiments may be implemented.

### DETAILED DESCRIPTION

The inventor has recognized and appreciated that improved techniques for identification of metabolites are desired in various applications, such as disease diagnosis and treatment, drug discovery, preclinical development, clinical development, and many others. Metabolites may be defined as small molecules that are involved in general metabolic reactions and that are biosynthesized by a cell, tissue or organism. Mass spectrometry is a high-throughput technique capable of generating large amount of mass spectrometry data. Mass spectrometry-based approaches are increasingly used for metabolite identification and characterization. However, conventional approaches to analysis of mass spectrometry data may be not sufficient for identification of metabolites using mass spectrometry data for a number of reasons.

Existing approaches to analyzing and interpreting mass spectrometry data, including tandem mass spectrometry (MS/MS), include manual interpretation of mass spectra and identification of molecules in a reference database using an exact mass of an ion. However, metabolites are chemically and biologically diverse and many remain unknown. A metabolite may therefore not be identified using any reference database, simply because no existing databases include mass spectrometry data on such a metabolite. Thus, a novel metabolite may not be identified in a sample, which may be a particular shortcoming when identification of that metabolite is critical for prompt diagnosis and treatment of a medical condition or in other applications.

Furthermore, a metabolite may have different MS/MS fragmentation patterns in different experimental conditions, which may be the case even if the same equipment is used for analysis of a sample. As another limitation of conventional approaches, when scores are computed indicating proximity between a metabolite in a sample and one or more metabolites in a reference database, the scores may depend on a type of instrument used to analyze the sample and are therefore not easily interpreted and may be not useful for analysis of data obtained using different platforms.

Manual interpretation of mass spectrometry data may be tedious and time-consuming and may therefore be not appropriate for applications where a timely identification of a metabolite is required (e.g., in disease diagnosis or drug discovery). In some scenarios, manual interpretation of mass spectrometry data may be not feasible—for example, investigation of metabolites present in yeast samples (e.g., S. cerevisiae or other) may result in generation of about 10000 spectra of precursor and product ion data that may require an excessively long time to analyze. Moreover, metabolite identification for drug discovery and development may be hampered by lack of tools for processing large volumes of mass spectrometry data which may include potential drug candidates.

Thus, while mass spectrometry techniques can generate large amount of data that may shed light on structural information about metabolites, existing approaches to analysis of mass spectrometric data are not capable of handling the data with sufficient speed and accuracy, and may not be able to identify novel metabolites.

Accordingly, the inventor has recognized and appreciated that improved techniques for identification of metabolites are desired to process data generated using mass spectrometry techniques. Moreover, the inventor has recognized and appreciated that such techniques may utilize product ion data obtained by fragmenting a precursor ion by tandem mass spectrometry, to reveal a structure of a metabolite.

Thus, some embodiments provide techniques for processing MS/MS data that may identify metabolites in a sample with improved accuracy, sensitivity and speed. The techniques may involve structural identification of a metabolite regardless of whether it has been previously identified and included in a reference database. A scoring approach may be utilized that allows determining a likelihood of a correct identification of a metabolite, with scores being computed so that they do not depend on techniques used to acquire the analyzed mass spectrometry data.

The described techniques may utilize both precursor and product ion data obtained by subjecting a sample to tandem mass spectrometry (MS/MS) analysis. The precursor and product ion data thus obtained is referred to herein as experimentally obtained or observed precursor ion data and experimentally obtained or observed product ion data.

The inventor has appreciated that precursor ion data may be computationally generated, or computed, and stored, for example, in a first database. The first database may store any type of information on metabolites, including an identifier of each metabolite, a precursor ion m/z value of the metabolite, and any other suitable information.

In some embodiments, the first database may be accessed to compare information stored in the first database with precursor ion data that is obtained experimentally by subjecting the sample to tandem mass spectrometry. The comparison may allow identifying candidate metabolites in the first database that have a mass-to-charge ratio (m/z), interchangeably referred to herein as mass, that matches a mass of the experimentally obtained precursor ion. A mass of a computed precursor ion as used herein is taken as “matching” when it differs from a mass of an experimentally obtained precursor ion by a less than a certain tolerance value. For example, masses may be considered matching when they differ by less than 0.1, less than 0.01, less than 0.001 or by any other suitable value. In some embodiments, masses are taken as matching when they differ, for example, by less than or equal to 5 parts per million (ppm).

Candidate metabolites identified in the first database may be further analyzed to determine metabolites of the candidate metabolites that are likely to be correct matches that may reveal a structure of the metabolite being identified. The inventor has recognized and appreciated that the further analysis may use product ion data computationally generated for each of the candidate metabolites.

Accordingly, in some embodiments, product ion data may be computationally generated, or computed, for metabolites stored in the first database and stored, for example, in a second database. In some embodiments, the second database may be accessed to compare information stored in the second database with product ion data that is obtained experimentally by subjecting the sample to tandem mass spectrometry.

The second database may comprise, for each precursor ion of a metabolite in the first database, computed product ion data comprising product ion fragments that are predicted to result from fragmenting the precursor ion using tandem mass spectrometry. The fragments may be computed based on cleavage rules according to which metabolites break at chemical bonds when undergoing tandem mass spectrometry, as are known in the art. For example, the fragments may be generated as described in Murphy, R. C. (2002), “Mass spectrometry of phospholipids: tables of molecular and productions,” Illuminati Press, 71 s.; McAnoy, A. M., Wu C. C., and Murphy, R. C. (2005) “Direct Qualitative Analysis of Triacylglycerols by Electrospray Mass Spectrometry Using a Linear Ion Trap,” J Am Soc Mass Spectrom, 16, 1498-1509; Hsu, F.-F., Bohrer, A., and Turk, J. (1998), “Formation of Lithiated Adducts of Glycerophosphocholine Lipids Facilitates their Identification by Electrospray Ionization Tandem Mass Spectrometry,” J Am Soc Mass Spectrom 1998, 9, 516-526; Domon, B. and Costello, C. E. (1988), “A systematic nomenclature for carbohydrate fragmentations in FAB-MS/MS spectra of glycoconjugates,” Glycoconjugate, 5, 397-409; Musharraf, S. G., Ali, A., Khan, N. T., Yousuf, M., Choudhary, M. I. and Atta-ur-Rahman (2013), “Tandem mass spectrometry approach for the investigation of the steroidal metabolism: Structure—fragmentation relationship (SFR) in anabolic steroids and their metabolites by ESI-MS/MS analysis,” volume 78, issue 2, pages 171-181; Lewell, X. Q., Judd, D. B., Watson, S. P., Hann, M. M. (1998), “RECAP—retrosynthetic combinatorial analysis procedure: a powerful new technique for identifying privileged molecular fragments with useful applications in combinatorial chemistry,” J. Chem. Inf. Comput. Sci., 38, 511-522; Kasper, P. T., Rojas-Chertó, M., Mistrik, R., Reijmers, T., Hankemeier, T., Vreeken, R. J. (2012), “Fragmentation trees for the structural characterisation of metabolites,” Rapid Commun. Mass Spectrom., 26, 2275-2286; Katajamaa, M. and Oresic, M. “Processing methods for differential analysis of LC/MS profile data” (2005), BMC Bioinformatics, 6:179, each of which is incorporated herein by reference in its entirety.

Different cleavage rules may be used to generate fragments for metabolites of different types. It should be appreciated that any suitable cleavage rules may be utilized to generate fragments for a metabolite, as embodiments are not limited in this respect.

For each candidate metabolite identified in the first database comprising product ion data, a set of computed product ion fragments may be retrieved from the second database comprising computed product ion data. The sets of computed product ion fragments may then be compared to a set of experimentally obtained product ion fragments resulting from fragmenting a precursor ion in the sample using tandem mass spectrometry. The comparison may include determining, for each set of computed product ion fragments, a number of fragments in the set that match the experimentally obtained product ion fragments. The larger the number of the matching product ion fragments in a set, the more likely the set is to reveal the structure of the metabolite. Accordingly, by employing the database of computationally generated product ion data, a novel metabolite may be identified in the sample with improved accuracy.

In some embodiments, a score may be computed for each set of computed fragments retrieved from the second database, the score indicating correlation between the set of computed fragments and the set of experimentally obtained fragments. To compute the score, for example, each fragment in a set of computed fragments matching a corresponding fragment in the set of experimentally obtained fragments may be assigned a weight based on a relative abundance of the experimentally obtained fragment. A score may thus be computed for each set of computed fragments based on weights assigned to fragments in that set. The scores may then be used to rank the sets of computed product ion fragments to indicate which set matches more closely the set of experimentally obtained product ion fragments and therefore may be used to identify the metabolite.

The described techniques may allow identifying a metabolite in a sample using mass spectrometry data with improved speed. While conventional approaches may require several days to complete the metabolite identification, the techniques in accordance with some embodiments may perform the identification in several hours or in a shorter amount of time.

The described techniques may be applied to identify any types of metabolites. As used herein, a “metabolite” is defined as a small molecule compound which is an intermediate or a product of metabolism having a molecular weight less than approximately 1 kilo Dalton (kDa).

The small molecule may be, for example, an organic compound. Non-limiting examples of compounds may include ahydrolyzable tannins, aliphatic acyclic compounds, aliphatic heteromonocyclic compounds, alkaloids and derivatives, aromatic heteromonocyclic compounds, aromatic homopolycyclic compounds, carbohydrates and carbohydrate conjugates, homogeneous metal compounds, homogeneous non-metal compounds, hydrolyzable tannins, inorganic compounds, lignans and norlignans, mixed metal/non-metal compounds, nucleosides, nucleotides, analogues of nucleosides, analogues of nucleotides, organic acids, organic acids derivatives, organic halides, organometallic compounds, organophosphorus compounds, tannins, and any other suitable metabolites.

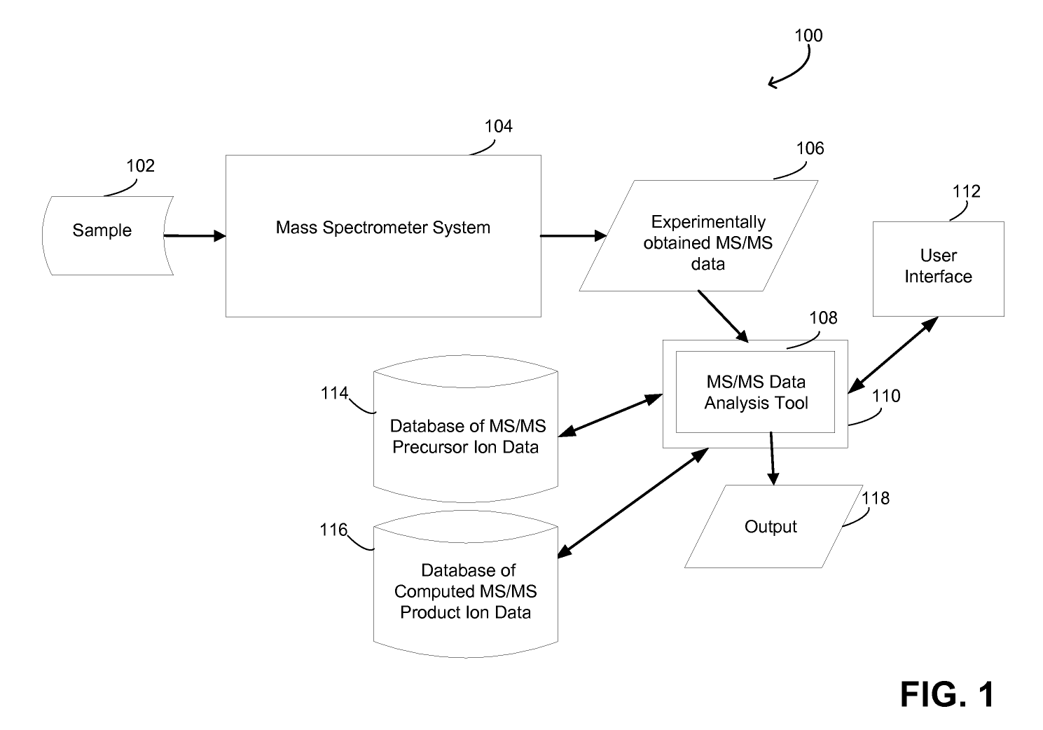

FIG. 1 illustrates generally a computing system 100 in which some embodiments of the invention may be implemented. As shown in FIG. 1, a sample 102 may be analyzed using a mass spectrometer system 104 which may comprise a tandem mass spectrometer. Sample 102 may comprise any complex mixture of molecules comprising metabolites of any suitable type. Sample 102 may be prepared using any suitable technique as is known in the art.

In some embodiments, sample 102 may be a biological sample obtained in any suitable manner from any subject. For example, sample 102 may be obtained directly from a subject or derived in any suitable manner—e.g., from blood, saliva, plasma, urine, other bodily fluids, tissues, or any sample which may comprise a metabolite compound, using any suitable technique of collecting a sample, as known in the art or developed in the future. “Subject” may include includes animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like. The subject may comprise a human subject. Though, in some embodiments, sample 102 may be obtained from bacteria, viruses, fungi or yeast. It should be appreciated that the techniques described herein are not limited to any particular organism or cell type, or any specific way of obtaining a sample from the organism or cell.

As shown in FIG. 1, regardless of the way in which sample 102 is obtained, sample 102 may be subjected to mass spectrometry analysis using mass spectrometer system 104, which may be a system of any suitable type. Mass spectrometer system 104 may comprise any suitable mass spectrometer that is a device that separates and quantifies ions based on their mass-to-charge ratios (m/z), as is known in the art. A mass spectrometer may comprise an ion source that ionizes a sample, a mass analyzer that separates the ions according their m/z values and an ion detector that detects ions and fragment ions.

In some embodiments, mass spectrometer system 104 may comprise a mass spectrometer that includes two or more mass analyzer that perform tandem mass spectrometry (MS/MS), which involves two or more stages of mass spectrometry selection, where fragmentation occurs between the stages. A precursor ion may first be selected and tandem mass spectrometry of that precursor ion may result in product ion fragments. Multiple stages of mass analysis separation can be performed with individual mass spectrometer components separated in space. Though, in some embodiments, mass spectrometer system 104 may comprise a single mass spectrometer with the mass spectrometry stages separated in time.

Mass spectrometer system 104 may include any number of mass spectrometers of any type. In some embodiments, mass spectrometer system 104 may include, in combination with one or more mass spectrometers, one or more separation devices, such as a gas chromatographer or a liquid chromatographer, that separate components in a mixture. In some embodiments, mass spectrometer system 104 may perform liquid chromatography-tandem mass spectrometry (LC-MS/MS). For example, mass spectrometer system 104 may perform electrospray ionization liquid chromatography (ESI-LC) tandem mass spectrometry (ESI-LC-MS/MS).

In some embodiments, mass spectrometer system 104 may perform a matrix-assisted laser desorption ionization (MALDI) mass spectrometry and may comprise, for example, a tandem time-of-flight (TOF/TOF) mass spectrometer. It should be appreciated, however, that embodiments described herein are not limited to any particular type of mass spectrometers. It should also be appreciated that mass spectrometer system 104 may comprise multiple mass spectrometers of any suitable type. In some embodiments, for example, a combination of mass spectrometers of different types may be utilized.

Mass spectrometer system 104 may analyze sample 102 using any suitable technique and, as a result, may output tandem mass spectrometry (MS/MS) data 106, which may comprise observed, or experimentally obtained, precursor and product ion data presented in any suitable format. Precursor ion data included in data 106 may comprise any suitable type of data, such as, for example a precursor ion m/z value, charge state, ion species (or adduct), precursor intensity, area, retention time, m/z values and corresponding intensities of higher isotopes, etc.

The experimentally obtained MS/MS data 106 may be received by MS/MS data analysis tool 108 which may perform processing for identifying one or more metabolites in sample 102 using the techniques in accordance with some embodiments. MS/MS data analysis tool 108 may be implemented in any suitable manner. For example, tool 108 may be implemented in computer-executable instructions encoded on a tangible computer-readable storage medium that, when executed by one or more processors, perform the described techniques for metabolite identification. The computer-executable instructions may be executed by one or more of any suitable processors of any computing device(s).

As shown schematically in FIG. 1, when implemented in computer-executable instructions, MS/MS data analysis tool 108 may be stored and executed on a computing device 110. Computing device 110 may be associated with mass spectrometer system 104 so that, in some embodiments, the device may receive MS/MS data 106 directly from mass spectrometer system 104. Though, MS/MS data 106 may be received by computing device 110 in any other manner. Moreover, in some embodiments, computing device 110 may be part of a system comprising mass spectrometer system 104 and any other suitable components.

In some embodiments, MS/MS data analysis tool 108 may be implemented as a web service as is known in the art. Computing device 110 configured to store and including MS/MS data analysis tool 108 may be a remote device which may receive MS/MS data 106 via a network, which may be a wireless network (e.g., the Internet). For example, computing device 110 may be a server computer configured to execute MS/MS data analysis tool 108, and a user may be able to access the server from a computing device via a network (e.g., the Internet) to process mass spectrometry data (e.g., sample 102), which may be obtained using any suitable tandem mass spectrometry technique. MS/MS data analysis tool 108 may be accessed via a browser executing on a user's device, such as a desktop, laptop, smartphone, tablet or any other type of computing device.

In other embodiments, one or more modules implementing at least part of functionality MS/MS data analysis tool 108 may be downloaded on a user device while one or more modules implementing remaining functionality MS/MS data analysis tool 108 may be executed on a remote device (e.g., a server). Though, in some embodiments, modules implementing the entire functionality of MS/MS data analysis tool 108 may stored in a memory of the user device and executed using one or more processors of the user device. It should be appreciated that the described techniques are not limited to any specific way of implementing MS/MS data analysis tool 108.

As shown in FIG. 1, system 100 may include a user interface 112 which may display various features of MS/MS data analysis tool 108, including prompts to a user to specify parameters and settings for executing MS/MS data analysis tool 108 and perform any other processing. User interface 112 may be presented on any suitable display—e.g., a display of computing device 110 or any other display.

MS/MS data analysis tool 108 may be implemented so that it may receive user input through a suitable user interface (e.g., user interface 112) as a user interacts with tool 108 to submit MS/MS data 106 to the tool, select various parameters and settings, view and edit results of metabolite identification, and perform any other operations. MS/MS data analysis tool 108 may present, on user interface 112 or any other interface, various visual and other types of features (e.g., audio) that may improve user experience in analyzing and interpreting mass spectrometric data and generating reports for various applications. For example, an experimentally obtained MS/MS spectrum may be annotated in a suitable manner so that one or more peaks in the spectrum are labeled with a corresponding metabolite compound determined to be represented by that peak.

FIG. 1 illustrates that system 100 may comprise a database of MS/MS precursor ion data 114 and a database of MS/MS product ion data 116. Databases 114 and 116 may be any suitable types of storage where data may be stored and maintained in any suitable manner. Databases 114 and 116 may be, for example, relational databases. Though, embodiments are not limited to any specific types of databases. Further, in some embodiments, databases 114 and 116 may be implemented in the same storage medium, or in any number of storage media.

Databases 114 may store MS/MS precursor ion data that may be obtained using any suitable mass spectrometry technique. It should be appreciated that, even though database 114 is referred to herein, by way of example, as a database storing MS/MS precursor ion data, metabolite data stored in database 114 may be obtained using any single MS based technique. Furthermore, data stored in database 114 may be computed (or computationally generated) or the stored data may be obtained experimentally. Similarly, database 116 may store computed (or computationally generated) data and/or data that is obtained experimentally.

In some embodiments, one or both of databases 114 and 116 may be included in MS/MS data analysis tool 108. For example, MS/MS data analysis tool 108, along with one or both of databases 114 and 116, may be loaded and executed on a user computing device which may receive experimentally obtained MS/MS data in a suitable manner. The user may be able to supplement information in databases 114 and 116 which additional information. In this way, the databases may be customized based on user preferences, particular types of metabolites, classes and subclasses of metabolites, and other factors.

Additionally or alternatively, one or both of databases 114 and 116 may be stored remotely, either along with, or separately from, MS/MS data analysis tool 108. For example, MS/MS data analysis tool 108 may be loaded on a computing device and databases 114 and 116 stored on a server may be accessed from the computing device by a user. In some embodiments, MS/MS data analysis tool 108 and the databases may be executed on a server and may be accessed by users via a network such as the Internet.

In some embodiments, databases 114 and 116 may store information on metabolites comprising small molecule compounds. Non-limiting examples of the compounds may comprise ahydrolyzable tannins, aliphatic acyclic compounds, aliphatic heteromonocyclic compounds, alkaloids and derivatives, aromatic heteromonocyclic compounds, aromatic homopolycyclic compounds, carbohydrates and carbohydrate conjugates, homogeneous metal compounds, homogeneous non-metal compounds, hydrolyzable tannins, inorganic compounds, lignans and norlignans, mixed metal/non-metal compounds, nucleosides, nucleotides, analogues of nucleosides, analogues of nucleotides, organic acids, organic acids derivatives, organic halides, organometallic compounds, organophosphorus compounds, tannins, and any other suitable metabolites.

MS/MS precursor ion data database 114 may comprise multiple entries each storing information relating to a metabolite compound and a precursor ion of the metabolite compound. Database 114 may store information on any suitable number of metabolites of any type, including metabolite compounds obtained from different sources, such as human, yeast, E. coli, plants, and any other sources.

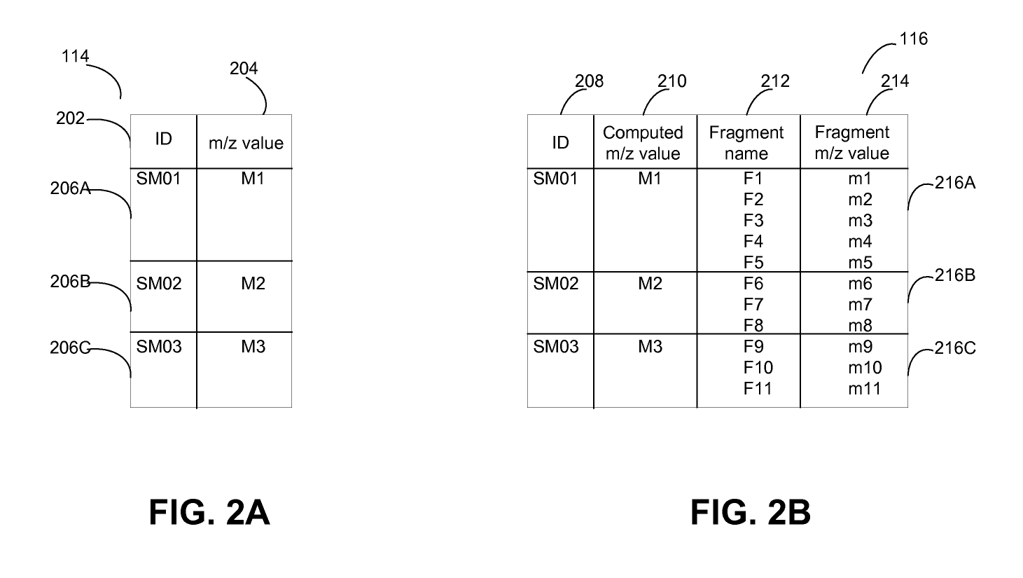

FIG. 2A illustrates schematically information stored in a portion of database 114 in accordance with some embodiments. As shown in FIG. 2A, database 114 includes at least a column storing an identifier (ID) 202 of a precursor ion of a metabolite compound and a column storing a mass-to-charge ratio (m/z) value 204 of the precursor ion. The identifier may be a unique identifier of a metabolite compound across databases 114 and 116, which may be of any suitable format and length. The identifier may be generated in any suitable manner.

Database 114 may include any suitable number of columns and rows (or entries). Though, database 114 may be stored in any suitable format. In the example illustrated in FIG. 2A, a row 206A stores an ID “SM01” and m/z value M1, a row 206B includes an ID “SM02” and m/z value M2, and a row 206C includes an ID “SM03” and m/z value M3. It should be appreciated that IDs and precursor ion m/z values are defined using the above notations by way of illustration only.

It should be appreciated that only a portion of database 114 is shown in this example, and database 114 may comprise any other information. Thus, each row in database 114 may include information comprising a metabolite compound's structure (e.g., a two-dimensional structure), name (e.g., a standard name of the metabolite), and molecular formula comprising elemental composition of the metabolite. The information may also comprise information on hierarchical classification of the metabolite, such as a super class which may be defined as a second level of hierarchical classification of the metabolite, with a first level being an indicator whether the metabolite is an organic or inorganic compound; a class or a subclass which may be defined as a third level of hierarchical classification of the metabolite; and/or information on hierarchical classification of any other type.

Further, the information stored in database 114 may comprise one or more identifiers of the compound used to store that compound in one or more other databases, which may be for example, public databases such as the Human Metabolome Database (HMDB), the Yeast Metabolome Database (YMDB), Kyoto Encyclopedia of Genes and Genomes (KEGG) or any other database. Any other suitable information may be stored in database 114, such as, for example, information on metabolic pathways, reactions, etc. It should be appreciated that embodiments are not limited to any specific type of information relating to a metabolite that may be stored in database 114.

Information stored in database 114 may be computed using any suitable technique. Additionally or alternatively, the information stored in database 114 may be experimentally obtained—for example, database 114 may store information on metabolites obtained by a third party. The information may also be obtained, at least in part, from other databases (e.g., public databases).

MS/MS product ion data database 116 may be linked to MS/MS precursor ion data database 114 and may include computationally generated product ion data. Database 116 may store, for each precursor ion of a metabolite compound stored in database 114, information on computed product ion fragments that are predicted to result from fragmenting that precursor ion using tandem mass spectrometry.

FIG. 2B illustrates schematically information stored in a portion of database 116, which may store information about fragment ions computationally generated for any number of metabolites of any suitable type. Database 116 may store information about computationally generated fragment ions of metabolites comprising small molecule compounds having a molecular weight of less than approximately 1 kilo Dalton (kDa).

For each metabolite compound in database 116, fragments may be computed based on cleavage rules according to which metabolites break at chemical bonds, as are known in the art. Different cleavage rules may be used to generate fragments for metabolites of different types.

Database 116 may include any suitable number of columns and rows (or entries). As shown by way of example in FIG. 2B, database 116 includes a column storing an identifier (ID) 208 of a precursor ion of a metabolite compound (ID) and a column storing an m/z value 210 of the precursor ion. ID 208 and m/z value 210 in database 116 correspond to the same ID 202 and m/z value 204 in database 114. An ID of a precursor ion of a metabolite compound may be a unique identifier that may be used as a key that links databases 114 and 116. Though, the databases may be linked in any other ways as embodiments are not limited in this respect.

FIG. 2B shows that each ID 208 and m/z value 210 in database 116 are further associated with one or more computed fragments that would result from fragmenting a precursor ion having ID 208 and m/z value 210. Thus, database 116 includes a column storing fragments 212 and a column storing respective m/z values 214 of the fragments 212. In this example, a row 216A in database 116 includes ID “SM01,” precursor ion m/z value M1, product ion fragments “F1,” “F2,” “F3,” “F4” and “F5,” each having a respective product ion m/z value m1, m2, m3, m4 and m5. Rows 216B and 216C include similar information for precursor ions “SM02” and “SM03.” It should be appreciated that IDs, precursor ion m/z values, product ion fragments and their m/z values are defined using the above notations by way of illustration only.

Further, it should be appreciated that only a portion of database 116 is shown in the example in FIG. 2B, as database 116 may comprise any other information. For example, database 116 may store, for each product ion fragment, that fragment's name which may be any type of identifier assigned to the fragment, structure (e.g., a two-dimensional structure of the fragment), molecular formula which is the elemental composition of the fragment, mass such as a monoisotopic mass of the fragment, and any other information.

It should be appreciated that databases 114 and 116 may store any type of information and may be organized in any suitable manner. It should also be appreciated that information stored in the databases may be stored in any suitable order, and FIGS. 2A and 2B illustrate the respective portions of the databases by way of example only. Further, in some embodiments, databases 114 and 116 may be organized as a single database or may be part of another database. Each of databases 114 and 116 may be updated by adding, removing or modifying information stored in the database, using any suitable technique as is known in the art.

Referring back to FIG. 1, MS/MS data analysis tool 108 may access databases 114 and 116 when processing experimentally obtained MS/MS data 106. In particular, experimentally obtained MS/MS data 106 may comprise precursor ion data, such as a precursor ion m/z value, and, first, database 114 may be accessed to identify one or more candidate metabolites with m/z values matching the m/z value of the experimentally obtained precursor ion data.

Once the one or more candidate metabolites are identified by querying database 114, database 116 may be then queried to identity computed product ion data associated with each of the candidate metabolites retrieved from database 114. The computed product ion data, such as a set of computed product ion fragments, retrieved from database 116 may be compared to experimentally obtained product ion fragments included in MS/MS data 106. The comparison may involve determining, for each set of computed product ion fragments, how many fragments in the set match fragments from the experimentally obtained product ion fragments. A fragment may be defined as “matching” when its computed m/z value differs from an m/z value of an experimentally obtained fragment by a value that is smaller than a certain tolerance value. In some embodiments, m/z values may be taken as matching when they differ by less than 0.1, less than 0.01, less than 0.01 or by any other value. In some embodiments, a numerical value (e.g., a score) may be computed indicating correlation between each set of computed product ion fragments and the experimentally obtained product ion fragments, based on a determined number of matching fragments and intensity of m/z values of respective experimentally obtained fragments. Scores computed for each set of computed product ion fragments may be used to as a measure of proximity indicating how closely each set matches the set of product ion fragments experimentally obtained by analyzing the sample using tandem mass spectrometry.

MS/MS data analysis tool 108 may provide results of the analysis of sample 102 as output 118, as shown schematically in FIG. 1. Output 118 may comprise, for example, one or more sets of computed product ion fragments each associated with a respective score indicating how closely that set matches the set of experimentally obtained product ion fragments. In embodiments in which more than one set of computed product ion fragments has been identified, in output 118, the sets may be ranked according to their respective scores. The ranking may indicate a likelihood that a particular set of computed product ion fragments may be used to correctly identify the set of experimentally obtained product ion fragments. In this way, output 118 may include results of determining a structure of the metabolite in the sample.

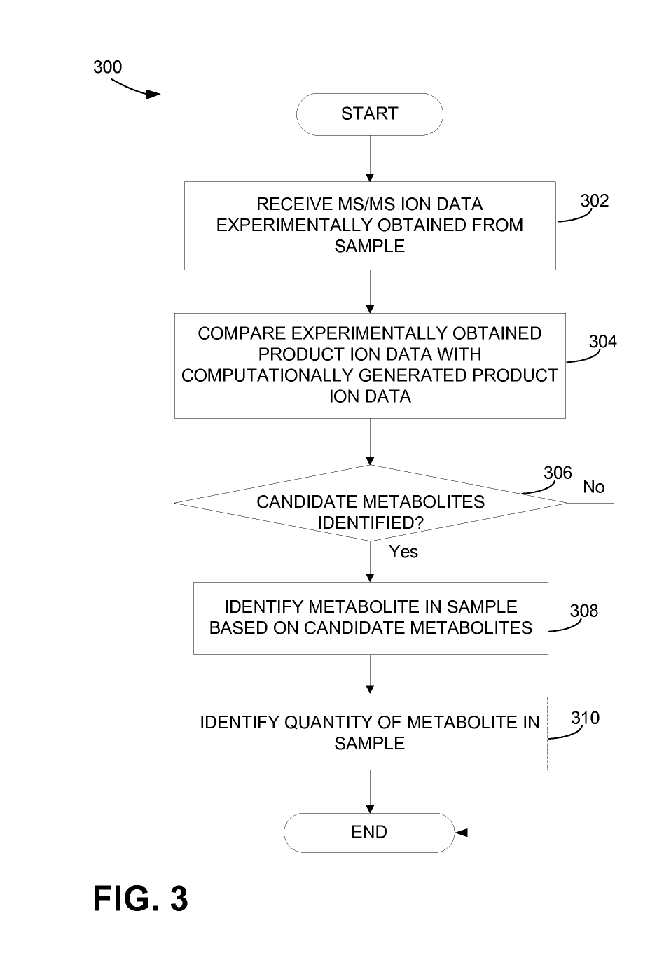

FIG. 3 illustrates generally a process 300 of identifying a metabolite in a sample using the described techniques which may include identifying a structure of the metabolite using product ion data. Process 300 may be implemented, at least in part, by MS/MS data analysis tool 108, or in any other suitable manner.

Process 300 may start at any suitable time—for example, when MS/MS data comprising precursor and product ion data experimentally obtained by analyzing a sample using tandem mass spectrometry is received, at block 302. A user may desire to analyze the data to determine whether the sample includes one or more metabolites, and to identify and quantify the metabolites in the sample.

The precursor and product ion data may be received in any suitable manner. For example, the data may be obtained directly from a mass spectrometer system (e.g., mass spectrometer system 104 in FIG. 1) used to analyze the sample. Additionally or alternatively, the data output from a mass spectrometer system may be manually submitted by a user—e.g., in scenarios where the described techniques are implemented on a remote computer (e.g., a server) accessed via a user's computer, the user may submit the data through a browser on the user's computer. In some embodiments, the precursor and product ion data may be received as “raw,” unprocessed data output from a mass spectrometer, and may then be processed in a suitable manner, as described in more detail below.

The experimentally obtained precursor and product ion data may be presented in any suitable format. In some embodiments, the data may be recorded as a document storing the precursor and product ion data in a suitable format. The format may be any type of a standard or proprietary format, as embodiments are not limited to any particular type of format. For example, the format may comprise mzXML, mzData, MS EXCEL, TEXT, .BAF, .YEP, .FID, .D, .CEF, .RAW, .LCS, .TXT (MSe format), .RAW, .WIFF, .T2D, or any other type of format.

Referring back to FIG. 3, regardless of the way in which the experimentally obtained precursor and product ion data is received and a format of the data, next, at block 304, experimentally obtained product ion data may be compared with computationally generated product ion data. The experimentally obtained product ion data may comprise product ion fragments generated by fragmenting a precursor ion having a first precursor m/z value using tandem mass spectrometry of the sample. The product ion data is output by a mass spectrometer as a mass spectrum comprising peaks each characterized by an m/z value and an intensity value, with each peak representing a respective fragment. Thus, each of the product ion fragments may be represented as a first product m/z value and an intensity value corresponding to the first product m/z value.

The comparison process at block 304 may include accessing a product ion data database (e.g., database 116 in FIG. 1) to identify sets of computationally generated product ion fragments that match the experimentally obtained product ion fragments. The computationally generated product ion fragments may be stored in the product ion data database in associated with an identifier of a precursor ion that is predicted to fragment into these fragments, with the precursor ion having a second precursor m/z value that matches the first precursor m/z value of the precursor ion that was fragmented to experimentally generate the product ion fragments.

Next, at decision block 306, it may be determined whether one or more candidate matching metabolites are identified based on the processing. The candidate matching metabolites may be identified when one or more sets of computationally generated product ion fragments match the experimentally obtained product ion fragments. A set from the sets of computationally generated product ion fragments may be identified as matching when a number of fragments in this set that match respective fragments in the experimentally obtained product ion fragments is above a certain threshold number. In some embodiments, when such matching fragments are identified in a set of computationally generated product ion fragments, a score may be computed for that set to indicate how closely the fragments in the set match the fragments in the experimentally obtained product ion fragments.

As shown in FIG. 3, if it is determined that no candidate metabolites are identified, process 300 may end.

If it is determined, at decision block 306, that the matching candidate metabolites are identified, process 300 may continue to block 308 where the metabolite in the sample may be identified based on the candidate metabolites, which includes identifying structure of the metabolite. The identification process may include generating an output comprising results of the identification. For example, the output may comprise one or more sets of computationally generated fragments that may be presented in association with the respective score computed for each set. In some embodiments, the sets of computationally generated fragments may be ranked in accordance with the scores, with the highest rank indicating the best match for the metabolite in the sample. Results may be presented to a user on a suitable user interface. In some embodiments, as shown at block 310 in FIG. 3, quantity of the identified metabolite in the sample may be determined using any suitable technique. In some embodiments, quantification of one o more metabolites identified in the sample 102 may be performed using an internal or external standard technique, as is known in the art. It should be appreciated, however, that embodiments described herein are not limited to any particular techniques for metabolite quantification.

Furthermore, it should be appreciated that process 300 may be executed continuously to identify any suitable number of metabolites in a sample or multiple samples. Process 300 may be used to identify metabolites with an improved speed and accuracy relative to conventional approaches.

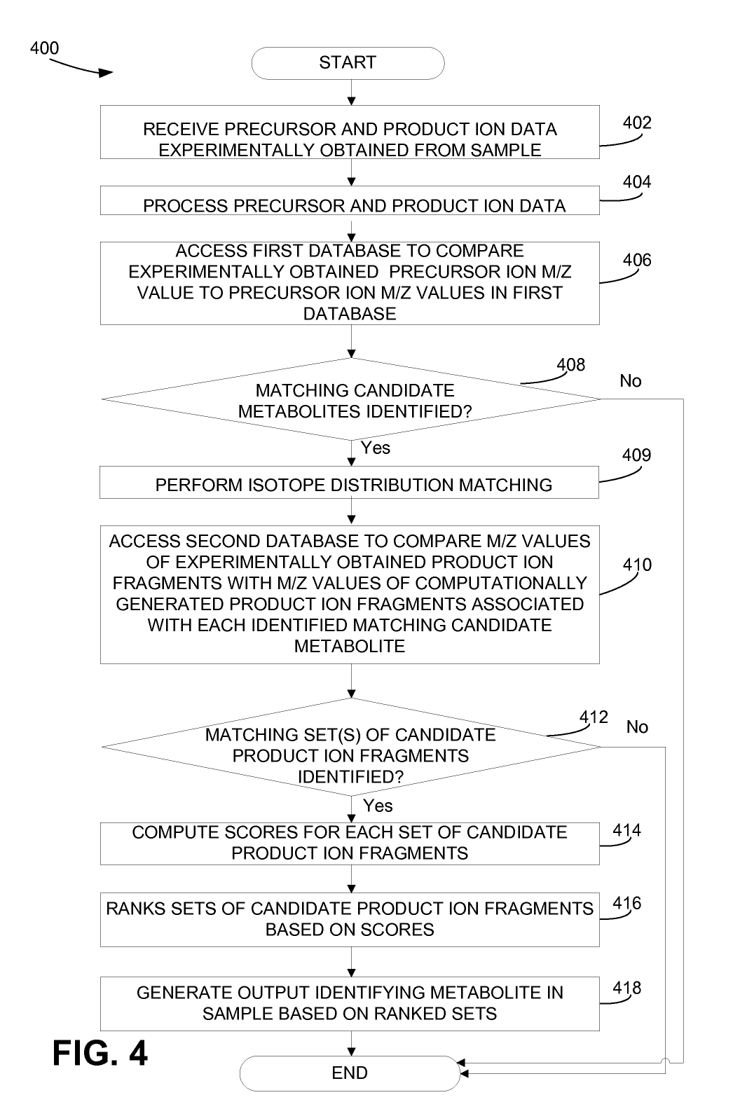

FIG. 4 illustrates in more detail a process 400 of identifying a metabolite in a sample using the techniques as described herein. Process 400 may be implemented by MS/MS data analysis tool 108 (FIG. 1), or in any other suitable manner. Process 400 may start at any suitable time—for example, when MS/MS data comprising precursor and product ion data experimentally obtained by analyzing a sample using tandem mass spectrometry is received, at block 402. In some embodiments, process 400 may start when an MS/MS data analysis tool is initiated based on user input or in any other manner.

In embodiments in which the experimentally obtained precursor and product ion data comprises raw data output directly from a mass spectrometer, the data may be processed, at block 404. The processing may comprise peak identification to generate a list of peaks corresponding to metabolite compounds, identifying isotopes from the list of peaks, noise removal, filtering/smoothing, alignment, gap filling, normalization, and any other suitable processing. In some embodiments, peaks may be identified based on a retention time and an m/z value. Though, any other suitable techniques may be substituted.

Furthermore, a statistical processing approach, such as, for example, a Principal Component Analysis (PCA) may be performed on the processed data in order to reduce data dimensions without compromising the information present in the data. It should be appreciated, however, that MS/MS data may be processed in any other suitable manner, including using any suitable techniques as are known in the art, as embodiments are not limited to any specific ways to process the MS/MS data.

In some embodiments, isotopic distributions, which are collections of peaks occurring from the same molecular compound but having different compositions in their atomic isotopes, may be analyzed as part of processing at block 404. In some embodiments, charge state of peaks in the precursors and product ion data may be identified, for example, by measuring the distance between two peaks in the spectrum. In this way, as an example, a charge state of a peak may be determined to be n, where n=1, 2, 3, . . . , if a distance between two peaks equals to 1.0033/n within an allowable tolerance, where the numerator is the approximate mass of a neutron. If the calculated distance between peaks does not match 1.0028/n or, as another example, 1.0033/n, then it may be determined that the charge state may not be identified. Though, any other suitable techniques may be substituted.

In some embodiments, processing at block 404 may involve ion species/adduct identification as is known in the art. An adduct may be any ion formed by adduction of an ionic species to a molecule being analyzed. The adduct identification may involve, for example, grouping experimentally observed m/z values according to a retention time tolerance. The retention time tolerance may be specified based on user input, for example. Within each group, m/z values may be sorted (e.g., in ascending order) and may be subtracted from each other. For mass spectrometry data acquired in a positive mode of operation of a mass spectrometer, if a resultant mass matches residue mass of adducts, such as, for example, Na, K, Li, NH₄, a smaller m/z may be assigned as a protonated ion, while a larger m/z may be assigned to the adduct according to the residue mass. Ion species in a negative ion mode of operation of a mass spectrometer may be identified in a similar manner. Though, it should be appreciated that embodiments are not limited to any specific way of ion species/adduct identification.

Regardless of the way in which the experimentally obtained precursor and product ion data is processed, the experimentally obtained precursor ion data may comprise, for each metabolite to be identified, at least an m/z value. The experimentally obtained product ion data may comprise, for each fragment ion, at least an m/z value and an intensity value (e.g., a relative intensity value).

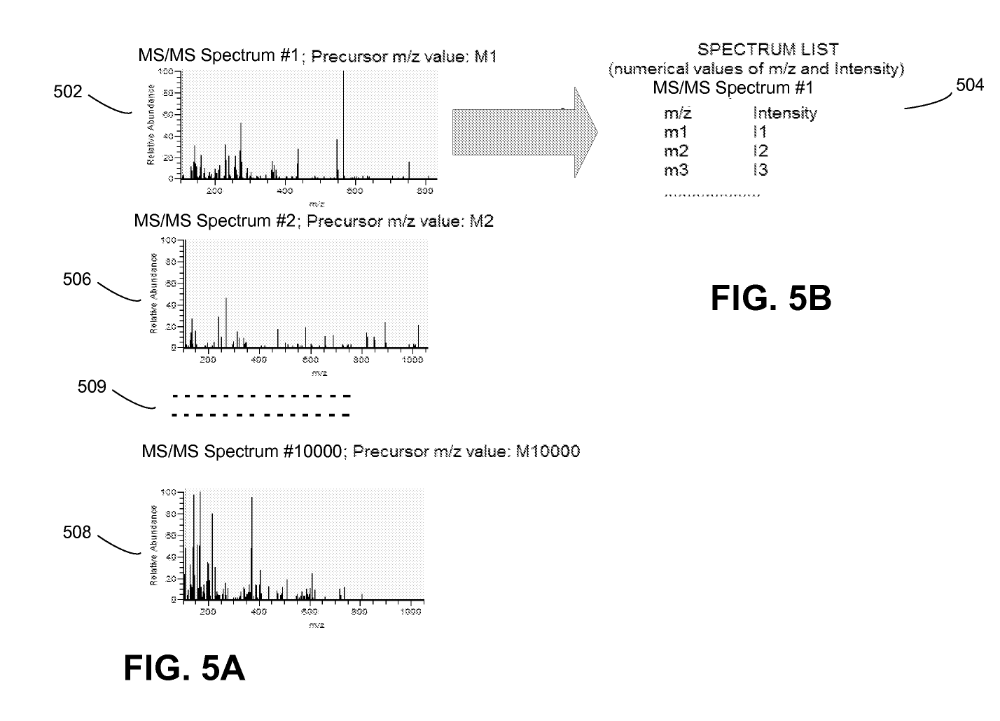

FIGS. 5A and 5B depict examples of product ion (MS/MS) spectra that may be analyzed using the described techniques. In the example illustrated in FIG. 5A, an MS/MS spectrum 502 (denoted as “MS/MS Spectrum #1”) is associated with an experimentally obtained precursor ion having an m/z value of M1. As shown in FIG. 5B, MS/MS spectrum 502 may be processed and represented as a spectrum list 504 comprising a set of experimentally obtained m/z values and corresponding intensity values. Spectrum list 504 may comprise information on any suitable number of peaks detected in MS/MS spectrum 502.

Mass spectrometry is a high throughput technique and a large number of MS/MS spectra may be processed by a mass spectrometer system when analyzing one or multiple samples. Thus, FIG. 5A illustrates by way of example that a large number of MS/MS spectra may be processed using a suitable technique to be then analyzed for the presence of a metabolite, which are shown, in this example, as a ten thousand of spectra. Thus, FIG. 5A shows an MS/MS spectrum 506 (denoted as “MS/MS Spectrum #2”) and an MS/MS spectrum 508 (denoted as “MS/MS Spectrum #10000”), with indicators “ . . . ” 509 indicating that there are other multiple spectra between spectra 506 and 508. It should be appreciated, however, that the described techniques are not limited to any specific number of MS/MS spectra that may be analyzed.

Referring back to FIG. 4, at block 404, a first database of precursor ion data (e.g., database 114) may be accessed to compare an m/z value of the experimentally obtained precursor ion to precursor m/z values of precursor ions stored in the database.

It may then be determined, at decision block 408, whether one or more matching candidate metabolites are identified based on the comparison at block 406. A match may be identified when a computed precursor m/z value stored in the precursor ion database differs from the m/z value of the experimentally obtained precursor ion by a value that is less than a tolerance value. The tolerance value may be, for example, 0.1, 0.01, 0.001, or any other suitable value.

The tolerance value may be selected in any suitable manner. In some embodiments, it may be selected based on user input—e.g., when a user interacts, through a user interface or in any other manner, with a tool such as, for example, MS/MS data analysis tool 108 implementing the described techniques. Additionally or alternatively, the tolerance value may be set automatically. If it is determined, at decision block 408, that no matching candidate metabolites are identified, process 400 may end.

In some embodiments, each matching candidate metabolite retrieved from the first database of precursor ion data may be associated with a respective set of product ion fragments stored in the second database of product ion data. For example, as shown in FIGS. 2A and 2B, a metabolite having an identifier SM01 and precursor ion m/z value M1 stored in database 114 may be associated with a set of fragments F1-F5 having corresponding m/z values m1-m5.

In some scenarios, identifying a matching candidate metabolite based on precursor ion data analysis may be sufficient to identify a metabolite in a sample. However, in many cases, the analysis of precursor ion data may not be adequate to accurately identify a metabolite in the sample. Different metabolites may have identical or similar masses and an analysis based on mass only may not provide a correct identification of a metabolite. Moreover, multiple matching candidate metabolites may be retrieved from the database of precursor ion data based on a precursor m/z value, and, without further analysis, it may not be feasible to discriminate between the multiple matching candidate metabolites to determine which of the candidates is the best match that can be therefore used to identify a metabolite in the sample.

Accordingly, the described techniques include further validation of the identified matching candidate metabolites. The validation process may result in selecting one or more of the identified matching candidate metabolites that are more likely candidates for identification of a metabolite in the sample. In this way, a number of the matching candidate metabolites that may be further analyzed may be reduced, which may reduce an overall amount of data to be processed and thus facilitate metabolite identification.

In some embodiments, process 400 may include optional processing at block 409 comprising isotope distribution matching. The isotope distribution matching may comprise comparing an isotope distribution pattern computationally generated for each matching candidate metabolite to an isotope distribution pattern experimentally obtained from the sample being analyzed. The isotope distribution patterns may be computed using any suitable technique as is known in the art, and may be stored in any suitable storage medium.

An indicator (e.g., a score) may be computed to indicate how closely the computationally generated isotope distribution pattern matches the experimentally obtained isotope distribution pattern. For example, a regression-based scoring approach may be used to identify a best match based on, for example, a coefficient of determination of a regression equation that provides an overall measurement of the goodness of fit of a regression line to the points. Though, the score may be computed in any suitable manner, as embodiments are not limited in this respect.

In embodiments in which the isotope distribution matching is performed and a score for each of the matching candidate metabolites is computed, a set of the matching candidate metabolites may be reduced by selecting more likely candidates based on the scores computed for the candidate metabolites. For example, when the computed scores range from 0 to 1, with 1 indicating the best match and 0 indicating the worst match, candidates assigned scores that are greater than 0. 5 may be selected from the candidate metabolites for further processing. It should be appreciated, however, that any type of indicators in any suitable range may be computed for the candidate metabolites, and any threshold value may be used to identify more likely candidates. Regardless of the way in which the indicators are determined, candidates selected based on the indicators may be further processed using product ion data.

In some embodiments, more likely candidates for metabolite identification may be selected from the set of candidate metabolites based on user input. A user interface of a suitable computing device (e.g., user interface 112 in FIG. 1) may be configured to receive user input specifying features that may be used to narrow down the set of candidate metabolites. For example, user input may be received to restrict further analysis of candidate metabolites based on a number of carbon or double bonds in a candidate metabolite, a biological source, a class, a sub-class and/or any other characteristic(s) of the candidate metabolites. For example, if a user has some knowledge regarding a source of the sample being analyzed (e.g., yeast), the user may indicate through the user interface that further processing using product ion data should be limited to candidate metabolites that are indicated as yeast metabolites in a database storing metabolite precursor ion data (e.g., database 114 or other database). It should be appreciated that any suitable characteristics of the candidate metabolites may be used to select metabolites for further analysis, in any suitable manner.

Regardless of the way in which more likely candidate(s) for structural metabolite identification may be selected from the set of candidate metabolites, in some embodiments, further validation of the identified matching candidate metabolites may be based on using product ion data computationally generated (or computed) for each of the matching candidate metabolites.

Referring back to FIG. 4, if it is determined, at decision block 408, that one or more matching candidate metabolites are identified, and after the optional act of isotope distribution matching at block 409, process 400 may follow to block 410 where a second database of product ion data (e.g., database 116) may be accessed to compare m/z values of experimentally obtained product ion fragments with m/z values of fragments in a set of computationally generated product ion fragments stored in the second database and associated with each of the candidate metabolite fragments. It should be appreciated, however, that the product ion data comprising computationally generated product ion fragments may be stored in any suitable storage, including database 114 or any other storage.

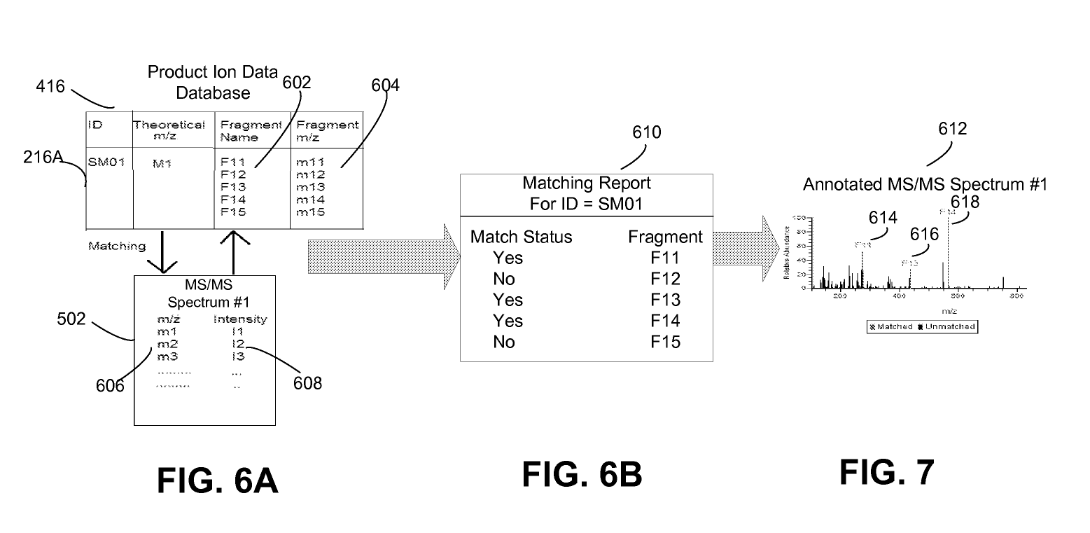

For example, continuing with the example shown in FIGS. 2A and 2B, a candidate metabolite having an identifier SM01 in database 114 may be one of the matching candidate metabolites. As shown in FIGS. 2B and 6A, information on this metabolite may be stored in row 216A in database 116 and may comprise a set 602 of candidate product ion fragments having respective m/z values 604.

Further, as shown in FIG. 6A, MS/MS spectrum 502 (#1) may be experimentally obtained product ion data comprising a set of product ion fragments represented by respective m/z values 606 and intensity values 608. Set 602 of candidate product ion fragments computationally generated for the candidate metabolite having the identifier SM01 and stored in database 116 may be compared to the experimentally obtained product ion data by comparing m/z values 604 with m/z values 606. Though, it should be appreciated that the set of experimentally obtained product ion fragments may be compared to the set of computationally generated product ion fragments in any suitable manner, using any suitable parameters. Accordingly, at decision block 412, it may be determined whether one or more matching sets of candidate product ion fragments are identified based on the comparison performed at block 410.

In the example shown in FIG. 6B, a result 610 of the comparison, shown by way of example as “Matching report,” may indicate which of fragments F11-F15 in set 602 match experimentally obtained fragments having m/z values 606. Result 610 indicates, for each of fragments F11-F15, whether a corresponding m/z value of the fragment matches (“Yes”) or not (“No”) an m/z value from m/z values 606. In this example, fragments F11, F13 and F14 are determined to be matching, while fragments F12 and F15 are determined to be non-matching. When more than one candidate metabolite is identified in database 114 and more than one respective set of matching sets of candidate product ion fragments is identified in database 116, the m/z values 606 may be compared to m/z values in each of such sets.

It should be appreciated that a match is indicated by “Yes” by way of example only, as the match may be any value determined based on a difference between m/z values. The match may be detected when an m/z value of a fragment in database 116 differs from an m/z value from the experimentally product ion data by a value that is smaller than a certain tolerance value (e.g., 0.1, 0.01, 0.001 or any other value). The tolerance value may be set based on user input. Additionally or alternatively, the tolerance value may be set automatically. It should be appreciated that the tolerance value may be any suitable value selected in any suitable way.

If it is determined, at decision block 412, that the one or more matching sets of candidate product ion fragments are identified, process 400 may continue to block 414 where a score may be computed for each set of candidate product ion fragments. In some embodiments, the score may be computed using intensity values from the experimentally product ion data, as discussed in more detail below.

At block 416, the matching sets of candidate product ion fragments may be ranked based on the computed scores. Next, at block 418, an output identifying a metabolite in the sample may be generated and process 400 may end. In some embodiments, the output may be based on the ranked matching sets of candidate product ion fragments. It should be appreciated, though, that the ranking may be optional and that the results of the identification of a metabolite in the sample may be presented in any other way.

The output generated at block 418 may comprise a list of identified metabolites along with corresponding computed scores, delta mass values (i.e., a difference between the experimentally obtained m/z value and the calculated m/z value for each metabolite), 2-D structure of the metabolite, class, sub-class, diagnostic (signature) ions that support the identification of the metabolite based on the computationally generated precursor and product ion data.

Furthermore, in some embodiments, information on the identified metabolite may be presented in association with experimental information such as a retention time, precursor intensity, area of the detected peak in the extracted ion chromatogram (XIC), and/or any other information. The information on the identified metabolite may be, for example, linked to the experimental information in a suitable manner so that a user can easily access the experimental information.

In some embodiments, the experimentally obtained MS/MS data, such as an MS/MS spectrum, may be annotated to indicate fragments in the spectrum for which a match has been found in a manner that differentiate these fragments from fragments for which no match has been found using the described techniques. For example, FIG. 7 illustrates that that MS/MS spectrum 502 may be presented as an annotated MS/MS spectrum 612 in which fragments matching computationally generated fragments F11, F13 and F14 are labeled with respective indicators 614, 616 and 618 indicating that matches were identified for these fragments. In the example illustrated, the indicators comprise color and a name of the fragment. Though, it should be appreciated that any types of indicators may be used to annotate MS/MS spectra, as embodiments are not limited in this respect. In some embodiments, the annotated spectra may be interactive—a user input may be received to modify a representation of the spectra in any suitable manner.

The output generated at block 418 may be presented in any suitable format. In some embodiments, the output may be generated as a report that may be stored, printed out, sent via an e-mail or in any other way, or manipulated in any other manner. The report may be a portable report that may be shared among different users. In some embodiments, the report may be presented in different formats.

In some embodiments, a user may specify a desired format for the output report. A tool (e.g., MS/MS data analysis tool 108) implementing the described techniques may receive, through a user interface or in any other manner, user input indicating the format for the output report, and generate the report in that format. The report may be generated in a configurable format such that a user may be able to modify or delete any information presented in the report, or add information to the report.

It should be appreciated that process 400 may be continuous and a large number of experimentally obtained MS and MS/MS spectra (e.g., as shown in FIG. 5A) may be processed to identify metabolites in complex mixtures. In some embodiments, the spectra may be processed in a batch mode.

Furthermore, in some embodiments, results of the metabolite identification may be used to supplement a precursor ion database (e.g., database 114), a product ion database (e.g., database 116), or any other suitable storage comprising data on metabolites. For example, if a particular metabolite is identified in a sample, information on this metabolite may be added to either or both of the databases. In this way, as more data is added by one or more users to the databases, the databases may be customized to particular applications.

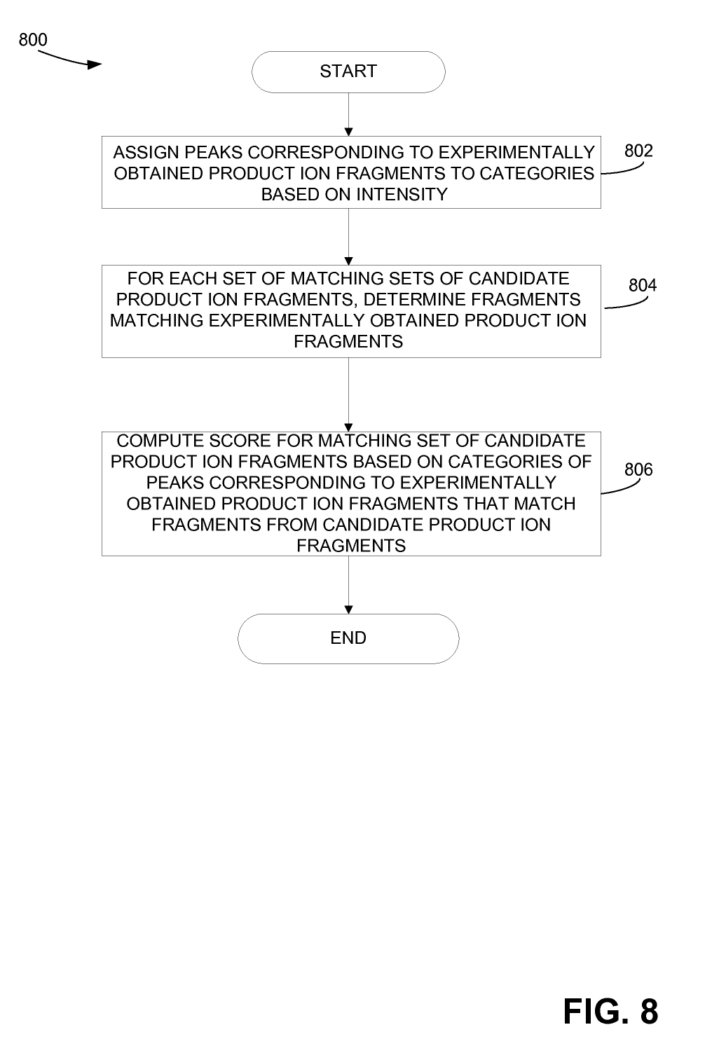

FIG. 8 illustrates a process 800 of computing a score for each set of matching sets of candidate product ion fragments matched against a set of experimentally obtained product ion fragments.

At block 802, peaks representing the experimentally obtained product ion fragments in an MS/MS spectrum may be assigned to categories based on their intensities. As an example, the peaks in the MS/MS spectrum may be assigned to four different categories based on the relative intensities of the base peak that is a peak with the highest intensity value in the spectrum. In this example, a first category may comprise peaks having relative intensities 80% and higher, a second category may comprise peaks having relative intensities equal or less than 80% but greater than 50%, a third category may comprise peaks having relative intensities equal or less than 50% but greater than 20%, and a fourth category may comprise peaks having relative intensities equal or less than 20%. However, it should be appreciated that the observed peaks may be assigned to any suitable number of categories based on any suitable cut-off values.

Next, at block 804, for each matching set of candidate product ion fragments, fragments matching the experimentally obtained product ion fragments may be determined. This may be performed, for example, using the processing at blocks 406-412 in FIG. 4.

Next, at block 806, a score may be computed for each matching set of candidate product ion fragments based on a number of fragments in the set that match the experimentally obtained product ion fragments and observed intensities of the experimentally obtained product ion fragments. In some embodiments, each fragment from the set may be assigned a weight based on a category of the corresponding matching fragments from the experimentally obtained product ion fragments. For example, continuing with the above example, fragments matching the experimentally obtained product ion fragments represented by peaks assigned to the first category may be assigned a weight of 1, fragments matching the experimentally obtained product ion fragments represented by peaks assigned to the second category may be assigned a weight of 0.7, fragments matching the experimentally obtained product ion fragments represented by peaks assigned to the third group may be assigned a weight of 0.5, and fragments matching the experimentally obtained product ion fragments represented by peaks assigned to the fourth category may be assigned a weight of 0.25. It should be appreciated, however, that the above weights are shown by way of example only, as embodiments are not limited to any particular weights that may be assigned to matching fragments based on intensity values.

The score may then be computed for each candidate metabolite, based on a combination (e.g., a sum) of the weighs assigned to each fragment computed for that metabolite that matches a respective observed fragment and a number of the matching fragments, or in any other suitable manner. In some embodiments, the computed scores may range from 0 to 100%, where 100% indicates that the candidate metabolite may be used to identify a metabolite in the sample with a high accuracy, whereas 0% indicates that the candidate metabolite is a false positive. For example, referring again to the example above, if all fragments computed for the candidate metabolite and matching respective peaks experimentally obtained by MS/MS analysis of the sample belong to the first group, the computed score for the candidate metabolite may be 100% score. Though, it should be appreciated that the scores may be computed using any other values having any suitable ranges indicating different likelihood that the candidate metabolite can be used to identify a metabolite.

The computed score may then be used to determine which of the matching candidate metabolites is the best match that may thus be used to identify a metabolite in the sample.

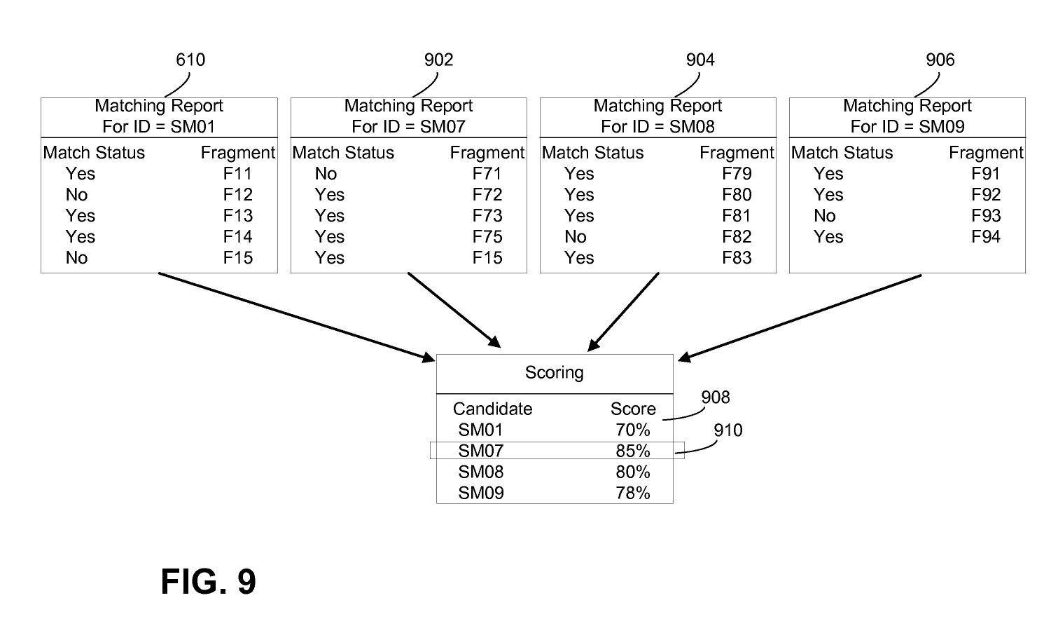

FIG. 9 illustrates that fragments computed for matching candidates SM01 610, SM07 902, SM08 904 and SM09 906 may be compared to the experimentally generated fragments to identify, for each of the matching candidates, matching fragments, as discussed above. Based on the matching, scores 908 may be computed for each of the matching candidates SM01 610, SM07 902, SM08 904 and SM09 906.

In this example, a score (85%) 910 computed for the candidate SM07 is the highest score among the computed scores. Thus, based on this score, the candidate SM07 may be used to identify the metabolite in the sample.

### Example I

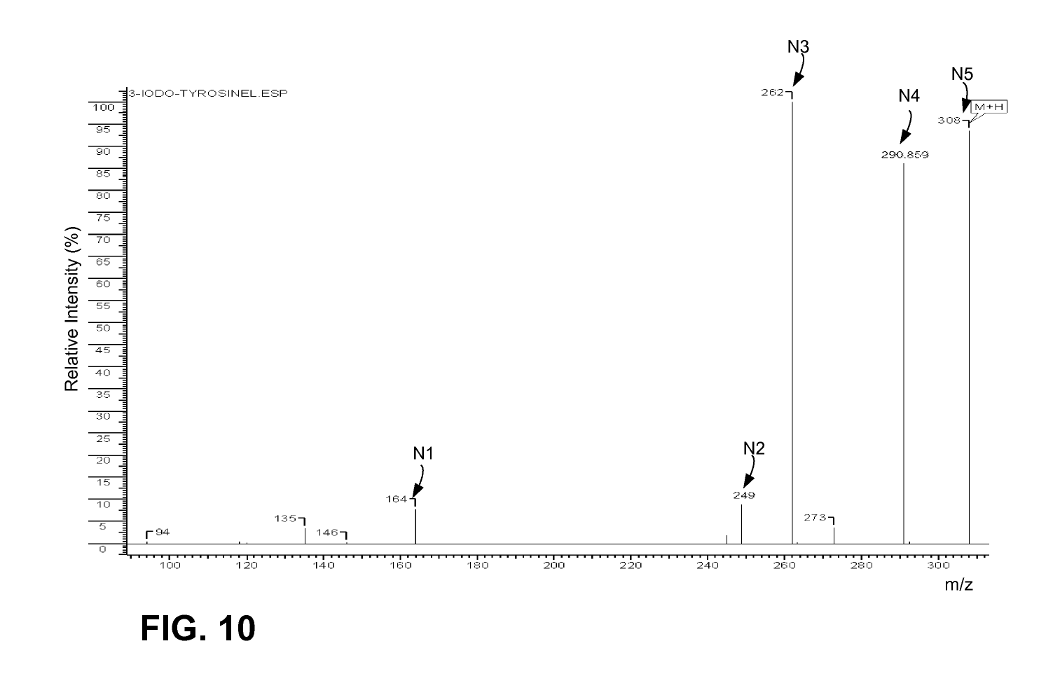

To demonstrate performance of the techniques as described herein, the inventor used the database of precursor ion data (e.g., database 114 in FIG. 1) and the database of product ion data (e.g., database 116 in FIG. 1) to identify a metabolite using MS/MS data on an Iodotyrosine compound with a precursor m/z value of 307.972, as shown in FIG. 10.

FIG. 10 illustrates product ion data obtained by subjecting a sample comprising the Iodotyrosine compound to tandem mass spectrometry. In this example, the product ion data comprises an MS/MS spectrum of the Iodotyrosine compound including five peaks, N1, N2, N3, N4 and N5, with relative intensities above a certain threshold (e.g., greater than approximately 7%, in this example). Information in Table 1 describes peaks N1-N5 detected in the MS/MS spectrum of the Iodotyrosine compound. In particular, Table 1 shows m/z values and a relative intensity (RI) value (in %) for each of the five peaks detected in the MS/MS spectrum of the Iodotyrosine compound, with each peak representing a respective fragment as shown in FIG. 10.

TABLE 1

| No. | m/z | RI (%) |
|---|---|---|
| 1 | 163.927 | 7.779 |
| 2 | 248.810 | 8.777 |
| 3 | 261.860 | 100 |
| 4 | 290.859 | 86.170 |
| 5 | 307.902 | 93.617 |

To assess the performance of the described techniques, first, the precursor ion data database, such as database 114, may be accessed to retrieve one or more candidate metabolites having an m/z value that is equal to or close to the precursor m/z value of 307.972 of the Iodotyrosine compound.

Table 2 illustrates that, based on comparison of the precursor ion experimentally obtained by analyzing the sample comprising Iodotyrosine compound with data stored in the precursor ion data database, four candidate metabolites may be identified. In this example, computed m/z values of the candidate metabolites from the precursor ion data database correlate with the m/z value of 307.972 of the experimentally obtained precursor ion within a tolerance value of 0.1.

Table 2 shows the four candidate metabolites identified by Human Metabolome Database (HMDB) identifiers (IDs). Each candidate metabolite is characterized by an identifier, name, molecular formula, structure, monoisotopic mass and computed precursor m/z (M+H).

TABLE 2

| | | | | | Computed |
|---|---|---|---|---|---|
| | | Molecular | | Monoisotopic | precursor m/z |
| ID | Name | formula | Structure | Mass | (M + H) |
| HMDB00021 | Iodotyrosine | C₉H₁₀INO₃ | 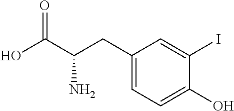 [SMILES: N[C@@H](Cc1ccc(O)c(I)c1)C(=O)O] | 306.970536611 | 307.978371 |
| HMDB14649 | Nitazoxanide | C₁₂H₉N₃O₅S | 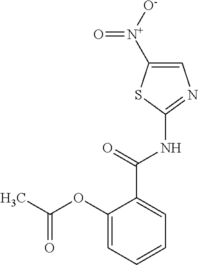 [SMILES: CC(=O)Oc1ccccc1C(=O)Nc1ncc([N+](=O)[O-])s1] | 307.026291103 | 308.034119 |
| HMDB14805 | Histamine Phosphate | C₅H₁₅N₃O₈P₂ | 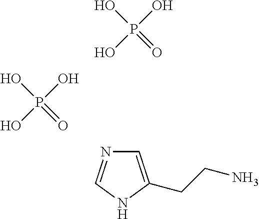 [SMILES: O=P(O)(O)O ; O=P(O)(O)O ; NCCc1cnc[nH]1] | 307.033437495 | 308.041268 |
| HMDB01202 | dCMP | C₉H₁₄N₃O₇P | 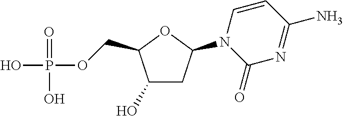 [SMILES: Nc1ccn([C@H]2C[C@H](O)[C@@H](COP(=O)(O)O)O2)c(=O)n1] | 307.056936329 | 308.064764 |

Each of compounds HMDB00021, HMDB14649, HMDB14805, and HMDB01202 is stored in association with a computed m/z value that differs from the m/z value of 307.902 of the peak in the experimentally obtained precursor ion by less than 0. 1.

Next, the product ion data database, such as database 116, may be accessed to retrieve product ion fragments computationally generated for each of compounds HMDB00021, HMDB14649, HMDB14805, and HMDB01202 retrieved from the database of precursor ion data. Each of the compounds may be associated with one or more sets of fragments stored in the product ion data database. Each of the sets may be compared to the experimentally obtained product ion data—the m/z values of the five fragment ions detected in the MS/MS spectrum of the Iodotyrosine compound. In this example, a computationally generated fragment ion from a set of computationally generated fragment ions is determined to be matching an experimentally obtained fragment ion when an m/z value of the computationally generated fragment ion differs from an m/z value of the experimentally obtained fragment ion by less than 0.2. In Tables 3-6 below, this difference is denoted by way of example as “Delta Mass.”

Relative intensity values of the experimentally obtained fragment ions for which matches are found in the product ion data database may be used to determine a likelihood that the matches can be used to identify a metabolite in the sample.

Table 3 illustrates results of comparing the experimentally obtained product data to computationally generated product data for the HMDB00021 compound.

TABLE 3

| Computed | Chemical | | | Matched experimental | Delta | |
|---|---|---|---|---|---|---|
| fragment | Composition | Mass | m/z | product ion | Mass | RI |
| 1 | C9H8O3I—H2O | 253.95 | 254.96 | | | |
| 2 | C8H9OI—H2O | 254.93 | 255.94 | | | |
| 3 | C8H8NIO | 260.97 | 261.97 | 261.86 | 0.11 | 100 |
| 4 | C9H8O3I—H2O | 271.96 | 272.97 | | | |
| 5 | C8H9OI—H2O | 272.94 | 273.95 | | | |
| 6 | C9O3H10NI—H2O | 288.96 | 289.97 | | | |
| 7 | C9H9O2IN | 289.97 | 290.98 | 290.86 | 0.12 | 86.17 |
| 8 | C8H9OI | 290.95 | 291.96 | | | |
| 9 | C9O3H10NI | 306.97 | 307.98 | 307.9 | 0.08 | 93.62 |

As shown in Table 3, three computationally generated fragment ions match respective fragments from the experimentally obtained product ions. In particular, computed fragments “3,” “7” and “9” match experimentally obtained fragments “3,” “4” and “5” (shown in Table 1), respectively. Further, relative intensities of all the experimentally obtained fragments “3,” “4” and “5”—100%, 86.17% and 93.617%, respectively, are above a certain threshold indicating that a relative intensity value is high. In this example, the threshold is 80%. Though, any other suitable threshold may be substituted.

Table 4 illustrates results of comparing the experimentally obtained product data to computationally generated product data for the HMDB14649 compound.

TABLE 4

| Computed | | | | Matched experimental | Delta | |
|---|---|---|---|---|---|---|
| fragment | Chemical Composition | Mass | m/z | product ion | Mass | RI |
| 1 | C3HN2SO2—2H2O | 92.96 | 93.97 | | | |
| 2 | C8H7O2—2H2O | 99.02 | 100.03 | | | |
| 3 | C3H2N3SO2—2H2O | 107.97 | 108.98 | | | |
| 4 | C3HN2SO2—H2O | 110.97 | 111.98 | | | |
| 5 | C8H7O2—H2O | 117.03 | 118.04 | | | |
| 6 | C3H2N3SO2—H2O | 125.98 | 126.99 | | | |
| 7 | C9H7O3—2H2O | 127.02 | 128.03 | | | |
| 8 | C3HN2SO2 | 128.98 | 129.99 | | | |
| 9 | C8H7O2 | 135.04 | 136.05 | | | |
| 10 | C9H8NO3—2H2O | 142.03 | 143.04 | | | |
| 11 | C3H2N3SO2 | 143.99 | 145 | | | |
| 12 | C9H7O3—H2O | 145.03 | 146.04 | | | |
| 13 | C9H8NO3—H2O | 160.04 | 161.05 | | | |
| 14 | C9H7O3 | 163.04 | 164.05 | 163.93 | 0.12 | 7.78 |
| 15 | C9H8NO3 | 178.05 | 179.06 | | | |
| 16 | C10H6O3SN3—2H2O | 211.99 | 213 | | | |
| 17 | C10H6O4N3S—2H2O | 227.99 | 229 | | | |
| 18 | C10H6O3SN3—H2O | 230 | 231.01 | | | |
| 19 | C10H6O4N3S—H2O | 246 | 247.01 | | | |
| 20 | C10H6O3SN3 | 248.01 | 249.02 | | | |
| 21 | C10H6O4N3S | 264.01 | 265.02 | | | |
| 22 | C12H9N3O5S | 289.02 | 290.03 | | | |
| 23 | C12H9N3O5S | 307.03 | 308.04 | 307.9 | 0.14 | 93.62 |

As shown in Table 4, two computationally generated fragment ions match respective fragments “1” and “5” from the experimentally obtained fragment ions. Only one of the matches (“23”), however, corresponds to the fragment ion “5” having a relative intensity value of 93.617 that is greater than the 80% threshold. A relative intensity value of the experimentally obtained fragment ion “1” matching a computed fragment “14” is low—7.78.

Table 5 illustrates results of comparing the experimentally obtained product data to computationally generated product data for the HMDB14805 compound.

TABLE 5

| Computed | Chemical | | | Matched experimental | | |
|---|---|---|---|---|---|---|
| fragment | Composition | Mass | m/z | product ion | Delta Mass | RI |
| 1 | C₅H₁₁N₃O₆P₂ | 271.01 | 272.02 | | | |
| 2 | C₅H₁₃N₃O₇P₂ | 289.02 | 290.03 | | | |
| 3 | C₅H₁₅N₃O₈P₂ | 307.03 | 308.04 | 307.9 | 0.14 | 93.62 |

Table 5 shows that a single computed fragment ion “3” matches the computationally generated fragment ion “5” that is associated with the relative intensity value greater than the threshold.

Table 6 illustrates results of comparing the experimentally obtained product data to computationally generated product data for the HMDB01202 compound.

TABLE 6

| | | | | Matched | | |
|---|---|---|---|---|---|---|
| Computed | Chemical | | | experimental | | |
| fragment | Composition | Mass | m/z | product ion | Delta Mass | RI |
| | C9H12O4N3—H2O | 62.96 | 63.97 | | | |
| 1 | C9H12O3N3—H2O | 78.96 | 79.97 | | | |
| 2 | H2O3P | 80.97 | 81.98 | | | |
| 3 | H2O4P | 96.97 | 97.98 | | | |
| 4 | H2O4P—H2O | 192.08 | 193.09 | | | |
| 5 | H2O3P—H2O | 208.07 | 209.08 | | | |
| 6 | C9H12O3N3 | 210.09 | 211.1 | | | |
| 7 | C9H12O4N3 | 226.08 | 227.09 | | | |
| 8 | C9H14O7N3P—H2O | 289.05 | 290.05 | | | |
| 9 | C9H14O7N3P | 307.06 | 308.06 | 307.9 | 0.16 | 93.62 |

Table 6 shows that a single computed fragment ion “9” matches the computationally generated fragment ion “5” that is associated with the relative intensity value greater than the threshold.

The above analysis shows that the HMDB00021 compound retrieved from the precursor ion data database is associated with a set of computed fragment ions in the product ion data database three of which are determined to match respective experimentally obtained fragment ions having high relative intensity values. As regards the three other candidate metabolites HMDB14649, HMDB14805, and HMDB01202, each of these metabolites is associated with a respective set of computed fragment ions comprising fragments only one of which is determined to match a respective experimentally obtained fragment ion having the high relative intensity. Accordingly, the HMDB00021 compound is the closest match to the experimentally obtained product ion data. The described techniques therefore correctly identify a metabolite in the sample as Iodotyrosine.

Techniques operating according to the principles described herein may be implemented in any suitable manner. Included in the discussion above are a series of flow charts showing the steps and acts of various processes that identify a metabolite in a sample. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

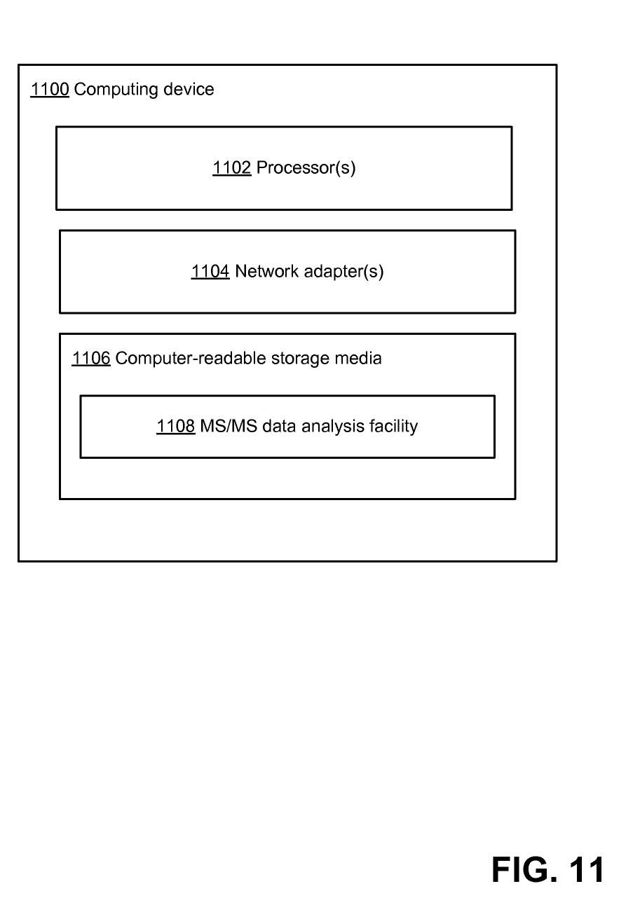

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 1106 of FIG. 11 described below (i.e., as a portion of a computing device 1100) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

FIG. 11 illustrates one exemplary implementation of a computing device in the form of a computing device 1100 that may be used in a system implementing techniques described herein, although others are possible. It should be appreciated that FIG. 11 is intended neither to be a depiction of necessary components for a computing device to implement the techniques for identifying metabolites in accordance with the principles described herein, nor a comprehensive depiction.

Computing device 1100 may comprise at least one processor 1102, a network adapter 1104, and computer-readable storage media 1106. Computing device 1100 may be, for example, a desktop or laptop personal computer, a personal digital assistant (PDA), a smart mobile phone, a server, or any other suitable computing device. Network adapter 1104 may be any suitable hardware and/or software to enable the computing device 1100 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media 1106 may be adapted to store data to be processed and/or instructions to be executed by processor 1102. Processor 1102 enables processing of data and execution of instructions.

The data and instructions stored on computer-readable storage media 1106 may comprise computer-executable instructions implementing techniques which operate according to the principles described herein. In the example of FIG. 11, computer-readable storage media 1106 stores computer-executable instructions implementing various facilities and storing various information as described above. Computer-readable storage media 1106 may store an MS/MS data analysis facility 1108 which may comprise, for example, computer-executable instructions that, when executed by processor 1102 perform processing in accordance with the techniques as described herein. In some embodiments, MS/MS data analysis facility 1108 may comprise computer-executable instructions that, when executed by processor 1102, implement MS/MS data analysis tool 108 (FIG. 1). MS/MS data analysis facility 1108 may also comprise one or both of a database of precursor ion data (e.g., database 114) and database of product ion data (e.g., database 116), or any other suitable storage comprising data used to identify a metabolite in accordance with some embodiments.

While not illustrated in FIG. 11, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure, and are intended to be within the spirit and scope of the principles described herein. Accordingly, the foregoing description and drawings are by way of example only.

What is claimed is:

1. A computer-implemented method of identifying a metabolite in a sample, the method comprising:
operating at least one processor to:
receive product ion data comprising observed product ion fragments generated by fragmenting a precursor ion having a first precursor m/z value using tandem mass spectrometry of the sample, each product ion fragment from the observed product ion fragments having a first product m/z value from a plurality of first product m/z values and an intensity value corresponding to the first product m/z value;
compare the plurality of first product m/z values to at least one set of second product m/z values of second product ion fragments of a molecule associated with a second precursor m/z value that matches the first precursor m/z value to identify at least one matching set of second product ion fragments; and
identify the metabolite based on the at least one matching set.

2. The method of claim 1, wherein the at least one matching set comprises a plurality of matching sets, and the method further comprises operating the at least one processor to:
for each matching set from the plurality of the matching sets:
determine matching fragments in the matching set having second product m/z values that match corresponding first product m/z values of the observed product ion fragments; and
compute a score for the matching set based on a number of the determined matching fragments in the set.

3. The method of claim 2, further comprising operating the at least one processor to:
rank the plurality of matching sets based on the scores computed for each of the matching sets.

4. The method of claim 2, wherein:
the score for the matching set is calculated based on a number of fragments in the matching set having second product m/z values that match corresponding first product m/z values of the observed product ion fragments and intensity values associated with the observed product ion fragments.

5. The method of claim 2, further comprising operating the at least one processor to:
associate each peak corresponding to a product ion fragment from the observed product ion fragments with a category based on an intensity of the peak, wherein:
computing the score for each matching set further comprises computing the score based on the categories of the peaks associated with first product m/z values that match corresponding second product m/z values of the determined matching fragments; and
identifying the metabolite based on the scores computed for the matching sets.

6. The method of claim 1, wherein:
the first precursor m/z value and the plurality of first product m/z values are obtained experimentally; and
the second product m/z values of the second product ion fragments and the second precursor m/z value are generated computationally.

7. The method of claim 1, wherein:
comparing the plurality of first product m/z values to the at least one set of second product m/z values of the second product ion fragments comprises accessing a database storing information on the second product ion fragments.

8. The method of claim 7, wherein:
the database stores information on a plurality of metabolites in a plurality of entries, each entry including a value corresponding to an identifier of a metabolite from the plurality of metabolites and information on a plurality of second product ion fragments that would result from fragmenting the metabolite using tandem mass spectrometry, the information on the plurality of second product ion fragments comprising one or more of the following: a value corresponding to an identifier of each product ion fragment, a representation of a molecular structure of the product ion fragment, a representation of a molecular formula of the product ion fragment, and a mass of the product ion fragment.

9. The method of claim 1, further comprising operating the at least one processor to:
selecting the molecule associated with the second precursor m/z value by:
comparing the first precursor m/z value to a plurality of second precursor m/z values each associated with a respective molecule from a plurality of molecules to identify the second precursor m/z value that matches the first precursor m/z value; and
selecting the molecule associated with the identified second precursor m/z value from the plurality of molecules.

10. The method of claim 9, further comprising operating the at least one processor to:
identify the precursor ion based on the molecule associated with the identified second precursor m/z value.

11. The method of claim 10, wherein selecting the molecule associated with the second precursor m/z comprising operating the at least one processor to:
access a database storing the plurality of second precursor m/z values each associated with a respective molecule.

12. The method of claim 1, wherein:
the metabolite comprises a small molecule.

13. The method of claim 1, wherein:
the metabolite has a molecular weight that is less than approximately 1 kilo Dalton (kDa).

14. The method of claim 1, further comprising operating the at least one processor to:
display product ion spectra representing the observed product ion fragments, each associated with a first product m/z value, so that at least one peak in the spectra having a first product m/z value that matches a corresponding second product m/z value from the at least one matching set is annotated using a second product ion fragment associated with the matching second product m/z value.

15. At least one non-transitory computer-readable storage medium storing computer-executable instructions that, when executed by at least one processor, perform a method of identifying a metabolite in a sample, the method comprising:
receiving product ion data comprising observed product ion fragments generated by fragmenting a precursor ion having a first precursor m/z value using tandem mass spectrometry of the sample, each product ion fragment from the observed product ion fragments having a first product m/z value from a plurality of first product m/z values and an intensity value corresponding to the first product m/z value;
comparing the plurality of first product m/z values to at least one set of second product m/z values of second product ion fragments of a molecule associated with a second precursor m/z value that matches the first precursor m/z value to identify at least one matching set of second product ion fragments; and
identifying the metabolite based on the at least one matching set.

16. The at least one non-transitory computer-readable storage medium of claim 15, wherein:
the first precursor m/z value and the plurality of first product m/z values are obtained experimentally; and
the second product m/z values of the second product ion fragments and the second precursor m/z value are generated computationally.

17. The at least one non-transitory computer-readable storage medium of claim 15, wherein:
identifying the metabolite comprises identifying a structure of the metabolite.

18. The at least one non-transitory computer-readable storage medium of claim 15, wherein:
comparing the plurality of first product m/z values to the at least one set of second product m/z values of the second product ion fragments comprises accessing a database storing information on the second product ion fragments.

19. The at least one non-transitory computer-readable storage medium of claim 15, wherein the method further comprises:
associating each peak corresponding to a product ion fragment from the observed product ion fragments with a category based on an intensity of the peak; and
calculating a score for each matching set of the at least one matching set based on the categories of the peaks of the observed product ions fragments associated with first product m/z values that match second product m/z values in the matching set;
ranking the matching sets based on the calculated scores; and
identifying the metabolite based on the ranking.

20. The at least one non-transitory computer-readable storage medium of claim 19, wherein:
the identification of the metabolite is based on metabolites associated with the matching sets.

21. A system comprising:
at least one processor; and
at least one storage medium having encoded thereon computer-executable instructions that, when executed by the at least one processor, cause the at least one processor to perform a method of identifying a metabolite in a sample, the method comprising:
receiving mass spectrum data obtained by analyzing the sample using a tandem mass spectrometer, the mass spectrum data comprising observed product ion fragments generated by fragmenting a precursor ion having a first precursor m/z value, each product ion fragment having a first product m/z value from a plurality of first product m/z values and an intensity value corresponding to the first product m/z value;
comparing the plurality of first product m/z values to at least one set of second product m/z values of second product ion fragments of a molecule associated with a second precursor m/z value that matches the first precursor m/z value to identify at least one matching set of second product ion fragments; and
identifying the metabolite based on the at least one matching set.

22. The system of claim 21, wherein:
the at least one matching set comprises a plurality of matching sets; and
the method further comprises:
for each matching set from the plurality of the matching sets:
determining matching fragments from fragments in the matching set having second product m/z values that match corresponding first product m/z values of the observed product ion fragments; and
calculating a score for the matching set based on a number of the determined matching fragments in the set.

23. The system of claim 22, wherein:
the score for the matching set is calculated based on a number of fragments in the matching set having second product m/z values that match corresponding first product m/z values of the observed product ion fragments.

24. The system of claim 21, wherein:
the first precursor m/z value and the plurality of first product m/z values are obtained experimentally; and
the second product m/z values of the second product ion fragments and the second precursor m/z value are generated computationally.

25. The system of claim 21, wherein:
comparing the plurality of first product m/z values to the at least one set of second product m/z values of the second product ion fragments comprises accessing a database storing information on the second product ion fragments.

26. The system of claim 25, wherein:
the database stores information on a plurality of metabolites in a plurality of entries, each entry including a value corresponding to an identifier of a metabolite from the plurality of metabolites and information on a plurality of second product ion fragments that would result from fragmenting the metabolite using tandem mass spectrometry, the information on the plurality of second product ion fragments comprising one or more of the following: a value corresponding to an identifier of each product ion fragment, a representation of a molecular structure of the product ion fragment, a representation of a molecular formula of the product ion fragment, and a mass of the product ion fragment.

27. The system of claim 21, wherein:
the first precursor m/z value and the plurality of first product m/z values are obtained experimentally; and
the second product m/z values of the second product ion fragments and the second precursor m/z value are generated computationally.

28. The system of claim 21, wherein the method further comprises:
identifying the precursor ion corresponding to the metabolite by:
comparing a first isotope distribution of the precursor ion to a plurality of second isotope distributions of the precursor ion to identify a matching isotope distribution; and
identifying the precursor ion based on the matching isotope distribution.

29. The system of claim 28, wherein:
the first isotope distribution is obtained experimentally; and
the plurality of second isotope distributions are obtained computationally.

30. A method of identifying a metabolite in a sample, the method comprising:
generating precursor ion data by analyzing the sample using a tandem mass spectrometer;
selecting a precursor ion corresponding to the metabolite based on the precursor ion data, the precursor ion having a first precursor m/z value;
fragmenting the precursor ion to generate observed product ion fragments each having a first product m/z value from a plurality of first product m/z values and an intensity value corresponding to the first product m/z value;
comparing the plurality of first product m/z values to at least one set of second product m/z values of second product ion fragments of a metabolite associated with a second precursor m/z value that matches the first precursor m/z value to identify at least one matching set of second product ion fragments; and
identifying the metabolite based on the at least one matching set.

31. The method of claim 30, wherein:
the first precursor m/z value and the plurality of first product m/z values are obtained experimentally; and
the second product m/z values of the second product ion fragments and the second precursor m/z value are generated computationally.

32. The method of claim 30, wherein:
the at least one matching set comprises a plurality of matching sets; and
identifying the metabolite comprises ranking the plurality of matching sets in a ranking order of each matching set of the plurality of matching sets is based on a number of fragments in the matching set having second product m/z values that match corresponding first product m/z values of the observed product ion fragments.

33. A method of generating a database of product ion fragments of metabolites, the method comprising:
for each metabolite of the metabolites, computationally generating a plurality of product ion fragments that would result from fragmenting a precursor ion of the metabolite by tandem mass spectrometry; and
storing information on the metabolites in a plurality of entries in the database, each entry including a value corresponding to an identifier of the metabolite and information on the plurality of product ion fragments comprising one or more of the following: a value corresponding to an identifier of each product ion fragment, a representation of a molecular structure of the product ion fragment, a representation of a molecular formula of the product ion fragment, and a mass of the product ion fragment.

34. The method of claim 33, wherein:
the metabolite has a molecular weight that is less than approximately 1 kilo Dalton (kDa).

35. A computing device comprising:
at least one processor; and
memory communicatively coupled to the at least one processor, the memory configured to store a data structure comprising a plurality of entries, wherein:
each entry from the plurality of entries stores first information on at least one product ion fragment of a metabolite that would result from fragmenting the metabolite using a tandem mass spectrometer; and
each entry from the plurality of entries stores second information on the following: a value corresponding to an identifier of each product ion fragment from the at least one product ion fragment, a representation of a molecular structure of the product ion fragment, a representation of a molecular formula of the product ion fragment, and a mass of the product ion fragment.

36. The computing device of claim 35, wherein:
the metabolite has a molecular weight that is less than approximately 1 kilo Dalton (kDa).

37. A computer-implemented method of identifying a metabolite in a sample, the method comprising:
operating at least one processor to:
receive precursor ion data on an observed precursor ion and product ion data on observed product ion fragments obtained by subjecting a sample to tandem mass spectrometry, the product ion fragments generated by fragmenting the precursor ion by subjecting the sample to the tandem mass spectrometry;
access a first database storing precursor ion data to identify at least one matching candidate metabolite that matches the observed precursor ion data;
for each candidate metabolite of the at least one matching candidate metabolite, access a second database storing computationally generated product ion data to retrieve product ion fragments in the second database that are computationally generated for the candidate metabolite;
compare the retrieved computationally generated product ion fragments to the observed product ion fragments;
based on the comparison, identify at least one metabolite of the at least one matching candidate metabolite that is associated with computationally generated product ion fragments matching the observed product ion fragments; and
identify the metabolite in the sample based on the at least one identified metabolite.
